(19) United States
(12) Patent Application Publication
(10) Pub. No.: US 2025/0207085 A1
(43) Pub. Date: Jun. 26, 2025

(54) Bioinks for 3D Extrusion Bioprinting, Methods of Making and Uses Thereof

(71) Applicant: McMaster University, Hamilton (CA)

(72) Inventors: Jose Moran-Mirabal, Dundas (CA); Mabel Barreiro Carpio, Hamilton (CA); David Gonzalez Martinez, Hamilton (CA); Julia Ungureanu, Caledon (CA); Mohammadhossein Dabaghi, Burlington (CA); Jeremy Hirota, Dundas (CA)

(21) Appl. No.: 18/846,459

(22) PCT Filed: Apr. 12, 2023
(86) PCT No.: PCT/CA2023/050499

§ 371 Date: Sep. 12, 2024

Related U.S. Application Data
(60) Provisional application No. 63/330,075, filed on Apr. 12, 2022.

(51) Int. Cl. C12N 5/00; B29C 64/118; B29K 105/00; B29K 105/24; B33Y 10/00; B33Y 70/00; C09D 7/43; C09D 7/65; C09D 105/00; C09D 135/02; C12N 5/077

(52) U.S. Cl. CPC C12N 5/0062; B33Y 70/00; C09D 7/43; C09D 7/65; C09D 105/00; C09D 135/02; B29C 64/118; B29K 2105/0005; B29K 2105/24; B29K 2995/0056; B33Y 10/00; C12N 5/0656; C12N 2513/00; C12N 2533/30; C12N 2533/76; C12N 2537/10

(57) ABSTRACT

This disclosure relates to the field of 3D extrusion bioprinting, and in particular, to compositions of bioinks including components that are typically non-extrudable or present low printability. The present bioink comprises a rheology modifier, such as a partially crosslinked polyacrylic acid, a crosslinking polymer, and, optionally, one or more additives. The bioink provides for the use of synthetic crosslinking polymers as well as modified or unmodified biopolymers, and exhibits good printability.

### FIELD

The present disclosure relates to the field of 3D bioprinting, and in particular, to compositions of bioinks for extrusion printing, methods of making and uses thereof.

### BACKGROUND

3D bioprinting is a rapidly evolving technology with exciting applications in tissue engineering because it can enable the controlled deposition of cells and biopolymers in customized designs that mimic the complexity found in natural environments. Among the different 3D bioprinting technologies, extrusion is one of the most popular in biofabrication due to its simplicity and broad applicability. The working principle of extrusion bioprinting is based on the formation of a continuous stream of bioink that is controllably pushed through a nozzle and then gelled or hardened, via crosslinking of the polymers present in the bioink, to obtain a 3D structure that is formed layer-by-layer. To achieve high printability, this process requires the bioinks to comply with strict rheological requirements to form continuous filaments that are deposited and crosslinked layer by layer to form the final structure. Typically, higher viscosities result in better printability, but high viscosity also requires higher pressures to extrude the ink, meaning higher shear stress, which can adversely affect cells suspended in the bioink and in turn their post-printing viability. Additionally, higher viscosities are usually achieved by working with high polymer concentrations, which is not ideal for tissue engineering. Dense polymer matrices can be detrimental to the cells after printing, preventing cell spreading, migration and proliferation. The compromise between printability and biological functionality remains one of the key challenges in the formulation of extrusion bioinks and is one of the reasons why a limited number of materials can be successfully printed and are actively being used in extrusion bioprinting, despite the large variety of biomaterials available.

Different approaches have been developed to print materials that lack ideal rheological properties but still obtain good printability. Recently, there has been increased interest in the development of multi-component bioink formulations as an attempt to obtain bioinks that combine the benefits of each constituent component. From the printing process perspective, assisted bioprinting strategies have been implemented, where additional materials are used as a sacrificial receiving matrix or as sacrificial inks. These strategies place less stringent rheological requirements on the bioinks and have been used successfully to print complex structures despite the use of low printability (i.e., low viscosity, slow cross-linking) bioinks. However, assisted bioprinting strategies lack the simplicity of direct bioprinting because after the construct is printed the sacrificial materials must be removed to recover the desired structure. This increases the complexity of the printing process and the time required to complete it, increases the amount of waste generated, and introduces the potential for deformation or rupture of the structure during the matrix removal procedure. 3D bioprinting via extrusion is a promising technology with many applications in the biomedical and tissue engineering fields. However, the evolution of this technology has been limited by the rheological constraints that the extrusion printing process imposes on the inks and the restriction of the printing parameters imposed by cell viability.

Currently, there is a need for a general strategy that introduces the proper rheological properties to a wide range of bioink compositions, including those containing synthetic polymers, or naturally occurring (native) or modified biopolymers, to enable bioprinting processes with fewer constraints than currently used processes.

### SUMMARY

The present disclosure provides a versatile bioink design strategy capable of producing a wide range of printable bioinks based on synthetic polymers, modified or unmodified biopolymers, that do not require the use of a support sacrificial matrix. A general strategy for extrusion printing of polymers that are typically non-extrudable or present low printability at low concentrations is provided. This strategy provides a bioink composition comprising a minimum of two components: i) a rheology modifier, which primarily determines the flow properties of the inks and the printability range, and ii) a crosslinking polymer, which influences the physical and biochemical properties of the final printed structure. Additives can also be included in the bioink composition.

Thus, in one aspect, a bioink composition is provided comprising a rheology modifier(s), a crosslinking polymer(s), and, optionally, one or more additives.

In another aspect, a method for extrusion bioprinting is provided, comprising: i) formulating one or more bioinks based on desired physical, chemical, and biochemical properties as described herein; and ii) printing the one or more bioinks onto a structure.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

Certain embodiments of the disclosure will now be described in greater detail with reference to the attached drawings in which:

### BRIEF DESCRIPTION OF THE DRAWINGS

FIG. 1—Extrusion 3D printing with high printability can be achieved for non-extrudable or low printable (bio)polymers using Carbopol (CBP) as a rheological modifier. A) Mesh-like constructs printed using poly(ethyleneglycol) diacrylate (PEGDA, 10%), Xanthan gum (XG, 1.7%), and agarose (AGA, 0.7%) without CBP. B) Cylinder and mesh-like constructs printed with PEGDA-CBP (10% PEGDA, 0.75% CBP, 0.4% LAP—lithium phenyl-2,4,6-trimethylbenzoylphosphinate), XG-CBP (1.7% XG, 0.5% CBP), and AGA-CBP (0.7% AGA, 0.5% CBP) inks. C) Quantitative assessment of the printability of inks composed of CBP 0.5%, CBP 0.75%, PEGDA-CBP (10% PEGDA, 0.75% CBP, 0.4% LAP), XG-CBP (1.7% XG, 0.5% CBP), and AGA-CBP (0.7% AGA, 0.5% CBP) in water.

FIG. 2—CBP-based inks present ideal rheological properties for extrusion bioprinting. A) Viscosity vs angular frequency curves for PEGDA-CBP (10% PEGDA, 0.75% CBP, 0.4% LAP), XG-CBP (1.7% XG, 0.5% CBP), and AGA-CBP (0.7% AGA, 0.5% CBP) inks showing shear thinning behaviour. B) Thixotropy assessment performed at two different shear rates for PEGDA-CBP (10% PEGDA, 0.75% CBP, 0.4% LAP), XG-CBP (1.7% XG, 0.5% CBP), and AGA-CBP (0.7% AGA, 0.5% CBP) inks. C) Viscosity vs angular frequency curves for GelMA-CBP (7% GelMA (gelatin methacrylate), 0.25% CBP, 0.5% LAP) ink showing shear thinning behaviour. D) Thixotropy assessment performed at two different shear rates for GelMA-CBP (7% GelMA, 0.25% CBP, 0.5% LAP) ink.

FIG. 3—The rheological properties of the inks can be controlled by varying the concentration of the rheological modifier (CBP). A) Influence of the concentration of CBP on G′ and G″ of inks containing PEGDA at a fixed concentration of 7.5%. B) Influence of the concentration of PEGDA on G′ and G″ of inks containing CBP at a fixed concentration of 0.75%. C) Influence of the concentration of CBP on G′ and G″ of inks containing GelMA at a fixed concentration of 5%. D) Influence of the concentration of GelMA on G′ and G″ of inks containing CBP at a fixed concentration of 0.25%. E) Influence of the concentration of CBP on the viscosity of inks containing GelMA at a fixed concentration of 5%. F) Influence of the concentration of GelMA on the viscosity of inks containing CBP at a fixed concentration of 0.25%.

FIG. 4—Extrusion 3D printing of CBP-based inks with crosslinkable (bio)polymers at varying concentrations showcase the high printability and tunable mechanical properties that can be achieved. A) Cylinder and mesh-like constructs 3D printed with inks containing PEGDA (5, 7.5, 10, and 20%), LAP (0.4%) and CBP (0.75%), or GelMA (1, 3, 5, and 7%), LAP (0.5%) and CBP (0.5%). B) Influence of the concentration of GelMA on G′ and G′ of the 3D printed crosslinked structures. C) Influence of the concentration of PEGDA on G′ and G′ of the 3D printed crosslinked structures.

FIG. 5—Use of CBP-based inks allows high fidelity extrusion 3D printing of modified and unmodified biopolymers that are non-extrudable or present low printability on their own. Cylinder and mesh-like constructs 3D-printed using XG-CBP (1.7% XG, 0.5% CBP), AGA-CBP (0.7% AGA, 0.5% CBP), GelMA-CBP (5% GelMA, 0.5% CBP, 0.5% LAP), SilkMA-CBP (5% SilkMA, 0.5% CBP, 0.1% LAP), and WPMA-CBP (7% WPMA, 0.5% CBP, 0.5% LAP) inks.

FIG. 6—Use of CBP-based inks allows high fidelity extrusion 3D printing of modified and unmodified (bio)polymers supplemented with a variety of additives. A) Constructs 3D printed using PEGDA-CBP (7.5% PEGDA, 0.75% CBP, 0.4% LAP) inks supplemented with cellulose nanocrystals (CNC) at a concentration of 0.3% or 1%. B) Constructs 3D printed using PEGDA-CBP (7.5% PEGDA, 0.75% CBP, 0.4% LAP) inks supplemented with fluorescent microparticles (FM) at a concentration of 0.04%. C) Constructs 3D printed using GelMA-CBP (3% GelMA, 0.5% CBP, 0.5% LAP) inks supplemented with CNC (0.1%), collagen (0.1%), or fluorescent microspheres (FM-0.04%). D) Constructs 3D printed using PEGDA-CBP (7.5% PEGDA, 0.75% CBP, 0.4% LAP) inks supplemented with human lung tissue-extracted extra cellular matrix (hlECM) at a concentration of 0.4%.

FIG. 7—Use of CBP-based inks allows high fidelity extrusion 3D printing of complex structures from (bio)polymers that are non-extrudable or present low printability by themselves. The hollow ghost structure was printed using PEGDA-CBP (10% PEGDA, 0.75% CBP, 0.4% LAP) ink, the solid ear structure was printed using AGA-CBP (0.7% AGA, 0.5% CBP) ink, and the hollow nose structure was printed using GelMA-CBP (5% GelMA, 0.5% CBP, 0.5% LAP) ink.

FIG. 8—Different cell lines can be grown on top and within crosslinked constructs made from CBP-based inks with and without additives. A) HBEC-6KT epithelial cells cultured on top of constructs made from PEGDA-CBP (7.5% PEGDA, 0.75% CBP, 0.4% LAP), PEGDA-CBP-Sucrose (7.5% PEGDA, 0.75% CBP, 0.4% LAP, 0.2M sucrose), or PEGDA-CBP-Collagen (7.5% PEGDA, 0.75% CBP, 0.4% LAP, 0.05% collagen) inks. B) 3T3 fibroblasts cultured within constructs made from PEGDA-CBP-Collagen (7.5% PEGDA, 0.75% CBP, 0.4% LAP, 0.05% collagen), PEGDA-CBP-CNC (7.5% PEGDA, 0.75% CBP, 0.4% LAP, 0.3% CNC), or GelMA-CBP (5% GelMA, 0.25% CBP, 0.5% LAP) bioinks. All images are fluorescence images where cells were labelled with Calcein AM dye (stains viable only cells). Scale bars represent 100 μm.

FIG. 9—Extrusion 3D bioprinting of CBP-based bioinks containing 3T3 fibroblasts and demonstration of viability within printed structures up to 14 days. A) Images of the mesh-like construct 3D-bioprinted using a GelMA-CBP (5% GelMA, 0.4% CBP, 0.5% LAP) bioink that included 3T3 fibroblast cells, and of the cells within the structure 14 days after printing. B) Images of structures that were 3D printed using a PEGDA-CBP (7.5% PEGDA, 0.75% CBP, 0.4% LAP) bioink that included 3T3 fibroblast cells 14 days after printing. All images were taken from samples stained with Calcein AM, which stains only viable cells. Scale bars represent 500 μm.

FIG. 10—Comparison of the viscosity of Carbomer 940 (CBM) and Carbopol ETD 2020 NF (CBP) when subjected to shear stress at different rates.

### DETAILED DESCRIPTION OF THE INVENTION

A bioink composition is provided comprising a rheology modifier, a crosslinking polymer and, optionally, one or more additives.

The rheology modifier is a substance that alters the rheological properties of the composition, e.g. viscosity, shear thinning properties and mechanical properties such as modulus, while also being thixotropic, i.e. on application of a force the composition becomes less viscous (flowable and easily poured), but rapidly returns to its equilibrium viscosity within a fixed time on removal of the force. Thus, the rheology modifier(s) promotes retention of the printed shape of the bioink. Suitable rheology modifiers for use in the present composition include biocompatible (e.g. non-toxic and cell compatible) polymers which render the bioink composition to have an equilibrium viscosity under low shear in the range of about 1-5000 Pas, and to exhibit thixotropic behavior. The rheology modifier may be a hydrogel (e.g. swellable in aqueous solutions) at physiological pH. In one embodiment, the polymer that forms the hydrogel may be partially crosslinked. In one embodiment, the rheology modifier is a polyacrylic acid (PAA) polymer, including polyacrylic acid homopolymers, copolymers and interpolymers and partially crosslinked polymers. The PAA polymer may, for example, have a viscosity in the range of about 1-300 Pa·s (0.5 wt % at pH 7.5) and/or a particle size in the range of 0.1-100 μm. Examples of suitable PAA polymers include, but are not limited to, PAA homopolymers that are partially cross-linked to varying degrees (e.g. comprising crosslinking in an amount of from about 1-50%), also referred to herein as PCPAA polymers; PAA copolymers with a C₁₀-C₃₀ alkyl acrylate; and PAA interpolymer comprising a PAA homopolymer or copolymer and a block copolymer of polyethylene glycol and a long chain alkyl acid ester. The PAA polymers may be crosslinked with any suitable crosslinker such as, but not limited to, allyl sucrose, allyl pentaerythritol, methylene bis-acrylamide, bis-acrylamide, and genipin.

In this strategy, a partially cross-linked polymer, in one embodiment a partially crosslinked polyacrylic acid (PCPAA, such as the commercially available Carbopols—CBPs, or carbomers, including homopolymers, copolymers and interpolymers), fulfill the role of the rheology modifier that allows the successful 3D printing of non-extrudable polymer, including biopolymer, hydrogels. PCPAA comprises a family of nontoxic, cell compatible synthetic polymers that are soluble in polar solvents, including water, in which the PCPAA particles can uncoil and swell to greater than 500 times their original size, such as 750 times or 1000 times their original size. At or about physiological pH, PCPAA polymers form transparent solid-like-liquids, even at concentrations as low as 0.1%, that present shear-thinning and thixotropic behavior with fast recovery (i.e. within less than 10 seconds, preferably less than 5 seconds, for example, within 4, 3, or 2 seconds, and most preferably within 1 second). They are also very stable under a variety of conditions, such as variations in temperature, and moderate variations in pH, i.e. they maintain their rheological properties within an acceptable amount of deviation (within a range of 10%). These are important rheological properties for extrusion bioprinting.

In an embodiment, the rheology modifier possesses a viscosity which correlates with its concentration such that a decreased concentration of the rheology modifier has a reduced viscosity rendering the rheology modifier to be tuneable with respect to viscosity. The incorporation of a tuneable rheology modifier in the present bioink renders the bioink also to be tuneable or adjustable with respect to viscosity. Rheology modifiers which are tuneable with respect to viscosity include partially crosslinked polyacrylic acid interpolymers.

The bioink is formed by combining the rheology modifier with a cross-linking polymer that can be crosslinked through physical or chemical mechanisms, such as temperature, ions, photo-irradiation, or chemical reactions, to form a hydrogel network. In one embodiment, the crosslinking polymer comprises synthetic crosslinking polymers. In another embodiment, the crosslinking polymer comprises unmodified or native crosslinking biopolymers, while in other embodiments, the crosslinking polymer comprises modified crosslinking biopolymers. The term “biopolymer” refers to polymers produced from natural sources either chemically synthesized from a biological material or entirely biosynthesized by living organisms. The term “unmodified” with respect to biopolymers refers to native biopolymers, while the term “modified” with respect to biopolymers refers to biopolymers which have been altered from their native form to include an entity that does not form part of the native molecule. The entity may itself be a naturally-occurring entity or a non-naturally-occurring entity. The entity may impart on the biopolymer a property which augments its performance within the bioink, and may be a sidechain entity, a transformation in native functional groups, or addition of new functional groups, a label, a molecule, e.g. protein or enzyme, a therapeutic or the like.

Examples of synthetic crosslinking polymers for use to make a bioink in accordance with the invention include, but are not limited to: poly(ethylene glycol) diacrylate (PEGDA), poly(oligoethylene glycol methacrylate) (POEGMA), poly(N-isopropylacrylamide (PNIPAm), polyacrylic acid (PAA), polyacrylamides (PAAm), poly(vinyl alcohol) (PVA), poly(ethylene glycol) (PEG), poly(ethylene oxide) (PEO), poly(2-hydroxyethyl methacrylate) (PHEMA) or a mixture thereof. Silicone is also an example.

Unmodified naturally occurring crosslinking biopolymers for use in the bioink include proteins, polysaccharides, polynucleotides and mixtures thereof. Examples include but are not limited to: collagen (Col), gelatin (Gel), hyaluronan (HA), silk fibroin (SF), whey protein (WP), glycogen (Gly), starch, xanthan gum (XG), agarose (AGA), cell-derived biopolymers such as fibrinogen, glycosaminoglycans, actin, tubulin, tissue-derived extracellular matrix (ECM) proteins, nucleic acids or a mixture of two or more of them.

Modified naturally occurring crosslinking biopolymers for use in the bioink include but are not limited to: acrylated, methylated or partially oxidized biomolecules such as proteins or polysaccharides. Examples include collagen methacrylate (ColMA), gelatin methacrylate (GelMA), hyaluronan methacrylate (HAMA), silk fibroin methacrylate (SilkMA), whey protein methacrylate (WPMA), glycogen methacrylate (GlyMA), agarose methacrylate (AGAMA), carboxymethyl cellulose (CMC), carboxymethyl cellulose methacrylate (CMCMA), or a mixture of two or more of them.

The selected rheology modifier and crosslinking biopolymer are combined under conditions appropriate to form a hydrogel suitable for use as a bioink in accordance with the invention. Appropriate conditions may include an increase or decrease in temperature to promote dissolution of the components and/or prevent premature gelling. The rheology modifier is combined with a sufficient amount of crosslinking polymer to yield a bioink that exhibits acceptable printability, for example, having adequate shape fidelity, shear thinning behaviour and which exhibit thixotropic behaviour. The rheology modifier may be incorporated within the bioink in amounts ranging from about 0.1 to about 10% by weight. The amount of rheology modifier incorporated within the bioink is selected to yield a bioink having properties appropriate for its utility. The amount of rheology modifier will also vary with the crosslinking polymer with which it is combined. Amounts of rheology modifier in the range of about 0.1-1% by weight, such as about 0.1-0.5% by weight, and preferably less than about 0.5% by weight, are utilized when combined with synthetic crosslinking polymers to achieve a bioink with acceptable printability and rheology, while a broader range of amounts of rheology modifier may be utilized when combined with modified or unmodified biopolymers.

The crosslinking polymer is incorporated in amounts ranging from about 0.1-50% by wt. As one of skill in the art will appreciate, the mechanical properties of the biomaterial ink can be tuned by varying the concentrations of the rheology modifier and/or the crosslinking polymer(s). In one embodiment, to achieve a stiffer or more rigid product, for example a product that mimics cartilage, greater amounts of the rheology modifier and/or the crosslinking polymer may be used, while lesser amounts of the rheology modifier may be used, or lesser amounts of the crosslinking polymer may be used, e.g. less than about 10% by weight, or 5% by weight or less of crosslinking polymer, to yield a softer product that provides an environment suitable for cells, and which maximizes cell viability during and subsequent to printing.

As used herein, the term “about” is meant to refer to the indicated value plus or minus an amount which is expected to result in a similar outcome, for example, an amount which is greater than or less than the indicated value by about 10%.

Crosslinking of the crosslinking biopolymer may be activated in a number of ways, depending on the crosslinking polymer utilized. Thus, crosslinking of the biopolymer may be activated by thermogelation, either by application of an increase or decrease in temperature; by ionic crosslinking, in which ions are utilized to facilitate crosslinking including metal ions such as calcium, iron, zinc, chromium titanium, zirconium, aluminum and the like introduced in salt or chelate form: or by photo-initiated crosslinking initiated by application of UV or visible light radiation in combination with a photoinitiator. Crosslinking may be conducted prior to printing with the bioink, during printing or subsequent to printing, depending on the bioink and crosslinking method used, as will be appreciated by one of skill in the art. The degree of crosslinking may also be controlled in order to provide a product having desired mechanical properties in the final product or construct. For example, the greater the degree of crosslinking, the greater the stiffness of the final product, e.g. crosslinking of 10% or greater will result in a stiffer product, while less crosslinking yields a softer product.

Printability is defined herein as L² (which is the total perimeter of the internal square openings of a mesh, squared) divided by nAₒ (the number of square openings times the ideal area of each square mesh opening when printed). The present bioink exhibits a printability of at least about 0.4, preferably greater than 0.4, e.g. at least 0.5, 0.6, 0.7, 0.8 or greater.

The present bioinks are also thixotropic. The term “thixotropic” as used herein with respect to the present bioink composition refers to a composition that exhibits a decrease in viscosity from the original equilibrium viscosity when exposed to shear stress, and exhibits rapid viscosity recovery from the decreased post-shear viscosity to the original (e.g. pre-shear) equilibrium viscosity within a finite period of time following cessation of shear stress (e.g. less than 10 seconds, and preferably less than 5 seconds or within 1 second to result in adequate printing fidelity). In this regard, viscosity recovery refers to at least about 50% recovery to pre-shear equilibrium viscosity, e.g. at least about 60-80% recovery, preferably at least about 90%-95% recovery to the original equilibrium viscosity and, more preferably, to recovery of viscosity to essentially the original equilibrium viscosity.

Regarding shear thinning behaviour, the bioink exhibits a decrease in its equilibrium viscosity on application of increasing shear rates. The decrease in viscosity refers to at least a 10-fold decrease, preferably at least 100-fold, and optimally at least 1000-fold decrease from the original equilibrium viscosity, when the shear rate is increased from, for example, from 0.1 to 100 Hz.

The present bioinks possess a viscosity which may be tuned as described to achieve a desired target viscosity suitable for the use for which the bioink is intended. Viscosity of the bioinks at a low shear rate (0.1-1 Hz) is in the range of about 1-5000 Pas in a pH range between 6-8.

The bioink composition may also incorporate one or more additives such as, but not limited to, biomolecules, e.g. collagen, cellulose, and sucrose; extracellular matrix (ECM) proteins; nanoparticles; microparticles; porogens (pore-forming materials); sensing probes; photoinitiators; luminescent reporter molecules; fibrillar materials; carbon nanotubes; nanowires; small molecules; dyes; mammalian, bacterial, or fungal cells; phages; viral particles; salts; non-ionic molecules to support cell survival; and ionic compounds. Additives may be incorporated within the composition to introduce desired properties or tune certain properties in the ink or the crosslinked material after printing.

Additives may be incorporated into the bioink during combination of the rheology modifier and crosslinking polymer, prior to crosslinking, or subsequent to crosslinking, or may be applied to the bioink subsequent to application of the ink onto a surface or mould, depending on the additive. For example, some additives, such as cells, may be affected by biopolymer crosslinking conditions. Thus, incorporation of the additive and crosslinking conditions (e.g., light irradiation wavelength, photo initiator concentration) are selected accordingly in order to preserve the additive, e.g. cell viability. In an embodiment, one or more different cell types can be cultured within the bioink and retain viability. In another embodiment, one or more different cell types can be cultured on the exterior of structures produced from a bioink and retain viability. In other embodiments, the one or more different cell types are distributed both on the exterior and interior of a structure produced from the bioink.

The bioink is printed using extrusion bioprinting, including 3D extrusion bioprinting, in a manner known in the art, to extrude continuous filaments of bioink (or the mixture of the bioink with additives such as cells or other biomolecules), layer-by-layer, either in parallel or sequence, to build or coat three-dimensional constructs for applications including but not limited to biomedical, tissue engineering, drug delivery, implants, biomimetics and the like. For example, the present bioinks may be used in tissue models such as those for cancer and angiogenesis, biomimetics for organs and appendages, or to induce differentiation of cells.

The 3D printed structures may be treated post-printing to induce a response to stimuli, including but not limited to, stimuli such as mechanical, magnetic, ionic, electric (voltage or amperage), light, temperature, pH, or oxygen concentration. Such post-printing treatment may be done in order to, for example, change the mechanical, chemical or biochemical properties of the structure in a spatially or temporally modulated fashion or to expose cells included inside or on top of the structure to external stimulation.

In an embodiment, the biomaterial ink can be used as an enveloping or support matrix for applications involving other organisms such as bacteria, plants and animals. The 3D printed structures can be prepared using one or more different biomaterial ink compositions.

The bioinks are designed to tune the mechanical properties of the final construct by varying the concentration of the chosen crosslinking synthetic or bio-polymer, allowing the printing of large and complex freestanding 3D structures made of soft hydrogels that present excellent flexibility and stretchability (in the range of about 10-1000% strain). Furthermore, it is shown that a multiplicity of cells can be cultivated on top of a printed structure prepared from the bioink or included in the bioink and printed within the soft hydrogels. Cells are shown to retain a high degree of viability (e.g. greater than 75% viability, such 80-90% viability, for at least 1 week, and preferably up to several weeks of culture. The versatility of the present bioink and the ability to maintain cell viability results in its utility for a variety of applications.

### Definitions

Unless otherwise indicated, the definitions and embodiments described in this, and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5-10% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically, biologically or physically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “biomolecule” as used herein refers to any substance produced by living organisms or cells including but not limited to growth factors, structural biopolymers, and extracellular matrix.

It will be understood that any component defined herein as being included may be explicitly excluded by way of proviso or negative limitation, such as any specific compounds or method steps, whether implicitly or explicitly defined herein.

The present disclosure relates to a new technology for extrusion 3D bioprinting of crosslinking synthetic polymers and modified or unmodified crosslinking biopolymers that are non-extrudable or present low printability. The bioinks presented are composed of one or more crosslinking synthetic or bio-polymers, one or more rheology modifiers (e.g., PCPAA), and optionally one or more additives. Including a PCPAA rheological modifier in the formulation improves the printability of the polymer(s) by introducing desired rheological properties (i.e. shear-thinning and thixotropic behaviour) and maintains these properties when the bioink is optionally mixed with additives. Different concentrations of the crosslinking polymer can be used to tune the mechanical properties of the printed scaffolds. Furthermore, one or more additives can be introduced while maintaining good printability. The technology herein described is compatible with bioprinting, including culture and growth of different cell lines on top and/or within the crosslinked hydrogels.

Embodiments of the invention are described in the specific examples that follow, which are not to be construed as limiting.

### EXAMPLES

### Example 1-Extrusion 3D Printing with Various (Bio) Polymers

To demonstrate the role of the PCPAA as a rheology modifier, formulations comprising the crosslinkable polymers, poly(ethylene glycol) diacrylate (PEGDA), xanthan gum (XG) and agarose (AGA) with and without Carbopol (CBP) were prepared. In the formulations without PCPAA, these (bio)polymers present low printability and the printing trials produced puddles without recognizable shapes that did not resemble the targeted models. PCPAA changes the rheological properties of all the inks introducing characteristics that are desirable for extrusion 3D printing like shear-thinning and thixotropic behavior. Inclusion of PCPAA as a rheological modifier allowed the formation of smooth and stable bioink filaments that resulted in 3D printed structures with high shape fidelity when the crosslinking biopolymer was crosslinked. Two models were used to assess printability: a cylinder and a mesh-like construct. The cylinder was used to evaluate the deformation of the material under its own weight, while the mesh structure was employed to quantitatively assess printability. Overall, PCPAA enabled extrusion 3D printing with all the tested (bio)polymers, which were otherwise non-printable, showing that PCPAA can be used as a broadly applicable rheology modifier in 3D printing ink design.

Specifically, PEGDA-CBP, XG-CBP, and AGA-CBP inks were prepared as described below, and were then printed with high printability and excellent fidelity to designed shapes using CBP as PCPAA rheology modifier.

PCPAA (Carbopol® EDT 2020 NFR, IMCD Canada Limited, Brampton, ON, Canada, also referred to as CBP, which is an interpolymer) stock gel solution was prepared by suspending 1.5% of the polymer in ultrapure water and then neutralizing with 25 wt % NaOH.

Xanthan gum ink (XG-CBP) was prepared by mixing 10 mL of a previously prepared 2.5% w/v XG with 5 g of 1.5% CBP. A control ink was formulated without CBP at the same concentration of XG.

To prepare the agarose-based ink (AGA-CBP), agarose was added to ultrapure water to prepare a 1% w/v solution, heated at 90-95° C. and stirred until fully dissolved. The agarose solution (10 mL) was then mixed with prewarmed CBP gel (1.5 wt %, 5 g), and both were kept at 60-70° C. to prevent the agarose from gelling. While vortexing the solution to mix the components, the flask was submerged at intervals in a water bath at 70° C. to keep the system warm. The ink was kept at 60° C. and used immediately for printing. A control ink was formulated without CBP at the same concentration of AGA.

PEGDA-based ink (PEGDA-CBP) was prepared by mixing 1 g of PEGDA with 5 g of 1.5% CBP, 3 mL of ultrapure water and 1 mL of 4% of lithium phenyl-2,4,6-trimethylbenzoylphosphinate (LAP). The formulation was vortexed until homogeneity was achieved. A control ink was formulated without CBP at the same concentration of PEGDA.

Cylinder and mesh-like constructs were printed to assess qualitatively and quantitatively the printability of the inks. The printer used was an Allevi 3 printer, equipped with 3 pneumatic heated extrusion heads that included photo-crosslinking illumination light emitting devices that emit light of 365 and 405 nm. The mesh-like structure was employed for quantitative measurement of the printability. A printability parameter (P) was defined as:

$$P = \frac{{L}^{2}}{16 {A}_{0}}$$

where L is the experimentally measured perimeter of the internal square openings of the mesh and A₀ their ideal (designed) area.

Constructs printed with XG-based inks were crosslinked by submerging the constructs in a 2.5 mM FeCl₃ aqueous solution for 30 min. Agarose based inks were printed while keeping the extruder at 60° C., and the material was crosslinked during the printing process while cooling down to room temperature. The PEGDA-based bioink was crosslinked during the printing process by keeping the printer blue lamp (405 nm) on during printing at 2.5-10% intensity. As will be appreciated by one of skill in the art, printing conditions will vary with the bioprinter being used. The bioprinter is essentially used according to manufacturer instructions.

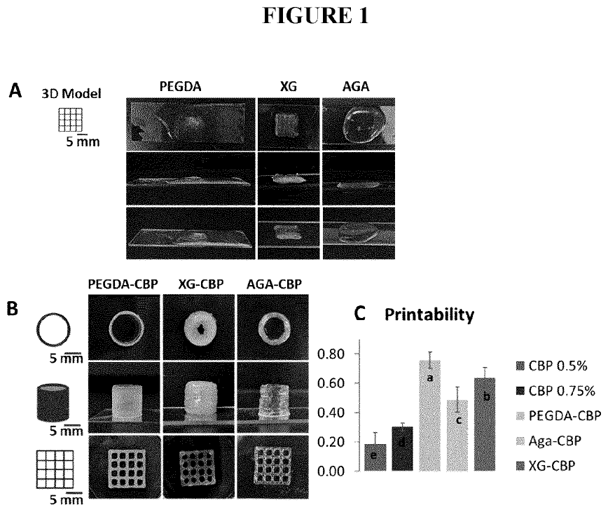

In all cases, the control inks without CBP did not exhibit good printability (FIG. 1A). It was not possible to recognize a defined structure that resembled the designed structure. These results confirmed that the polymers are not printable by themselves, and a rheology modifier must be added to be able to print constructs with good printability and high shape fidelity. CBP solutions on their own showed good extrusion capabilities, but the resulting structures deformed over time, losing fidelity to the designed structures. Incorporating crosslinking polymers into the CBP ink formulation dramatically improved the printing quality by providing a gel with the mechanical support that is required to retain the shape after the filaments were deposited (FIG. 1B). In addition, the qualitative assessment of the printability confirmed the key role that CBP plays in the inks, indicating a notable improvement of the (bio)polymer-CBP inks in comparison with CBP inks or the (bio)polymer by itself without the CBP (printability equal to 0, not included in the graph) (FIG. 1C). In all cases, acceptable printability (P) was considered to be at least about 0.4, and preferably 0.5 or greater.

### Example 2—PCPAA Tunes the Rheological Properties of the Bioinks

The rheological properties of the inks were measured at different concentrations of the PCPAA (CBP) to confirm its role as the primary component that determines the rheological behavior of the inks. Changes in CBP concentration significantly impacted the rheological properties of the inks, changing their behaviour from fluid liquids to liquid-like-solid gels. Higher PCPAA content increased the storage and loss modulus, and viscosity of the formulations.

Formulations containing CBP (0.1%-1.5%) and PEGDA (1-40%) or GelMA (0.5-20%) were produced. The printability of the PEGDA-PCPAA and GelMA-PCPAA biomaterial inks was assessed using cylinder and mesh-like structures. All the ink formulations presented excellent printability. The introduction of PCPAA enabled the printing of (bio)polymers in a broad range of concentrations, allowing the mechanical properties of the printed structures, including elastic modulus and elasticity, to be tuned. Increasing the PEGDA or GelMA concentration leads to higher mechanical modulus values. Higher (bio)polymer concentration will lead to a higher crosslinking density; therefore, the material will be mechanically stronger and less elastic.

Specifically, PEGDA-CBP inks with different percentages of CBP (0.25, 0.5, 0.75, and 1%) and fixed 7.5% of PEGDA, as well as inks with varying percentages of PEGDA (2.5, 5, 7.5, 10, 15, and 20%) and fixed 0.75% CBP, were formulated according to the procedure described in Example 1. XG-CBP (1.7% XG, 0.5% CBP) and AGA-CBP (0.7% AGA, 0.5% CBP) inks were formulated according to the procedure described in Example 1. GelMA inks were obtained by mixing the proper volume of 15% GelMA solution pre-warmed to 60° C. with the appropriate amount of 1.5% CBP and 4% LAP. Three inks with fixed 5% GelMA concentration and 0.1, 0.25, and 0.5% CBP were produced. In addition, three inks with fixed 0.25% CBP concentration and 3, 5, and 7% GelMA were produced. The final concentration of LAP in all GelMA-CBP inks was 0.5%.

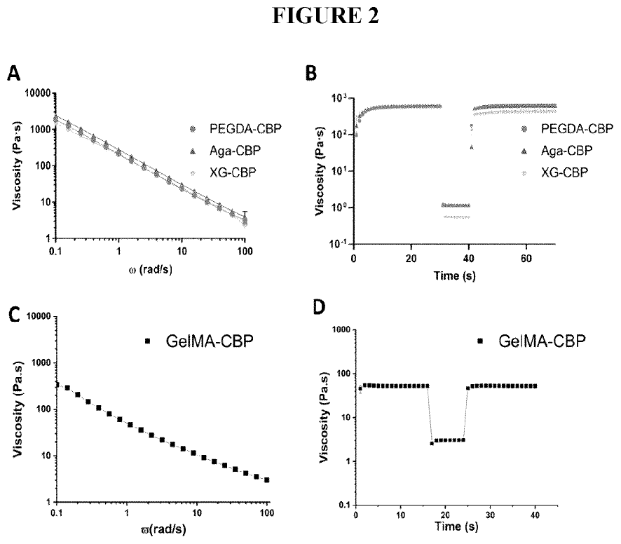

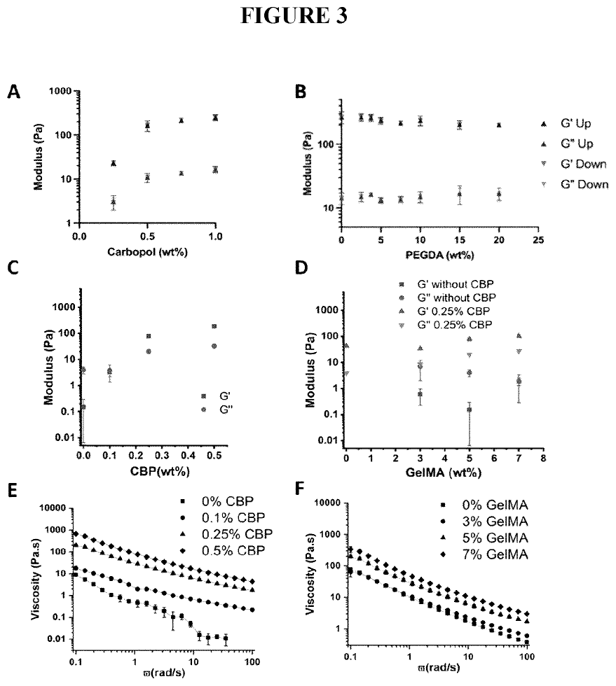

All the formulated bioinks presented the desired rheological properties, meaning shear thinning behavior (FIG. 2A, C) and thixotropy (FIG. 2B, D). FIG. 3A-B shows the influence of CBP and PEGDA percentages on the storage and loss modulus (G′ and G″) of PEGDA-CBP inks. Changes in CBP concentration significantly impacted the rheological properties of the bioinks, where higher content increased the modulus until it plateaued at concentrations higher than 0.5%. On the other hand, PEGDA concentration can be increased almost 8-fold, from 2.5 to 20 wt %, without significantly changing the rheological properties of the inks.

The influence of CBP in the properties of the ink was also explored by comparing G′, G″, and viscosity of GelMA inks with and without CBP (FIG. 3C-F). When CBP was added to GelMA, the viscosity and G′ of the formulation increased, showing that CBP is the main factor that determines the rheological properties of the ink. Viscosity and G′ of the inks increase at higher CBP and GelMA concentrations, however, the influence of CBP was larger than the impact of increasing GelMA concentration. Overall, these results demonstrate that CBP plays a dominant role in the rheological properties of the inks, which can be tuned by varying the concentration of the PCPAA modifier.

### Example 3—Extrusion 3D Printing with Bioinks Having Varying Crosslinking (Bio)Polymer Content

PEGDA-CBP inks with different percentages of PEGDA (5, 7.5, 10, and 20%) and fixed 0.75% of CBP were formulated according to the procedure described in Example 1. GelMA-CBP inks with different percentages of GelMA (1, 3, 5, and 7%) and fixed 0.5% of CBP were formulated according to the procedure described in Example 2. The inks were 3D printed according to the procedure described in Example 1. The GelMA ink was printed at 37° C. The printability was determined according to the procedure described in Example 1.

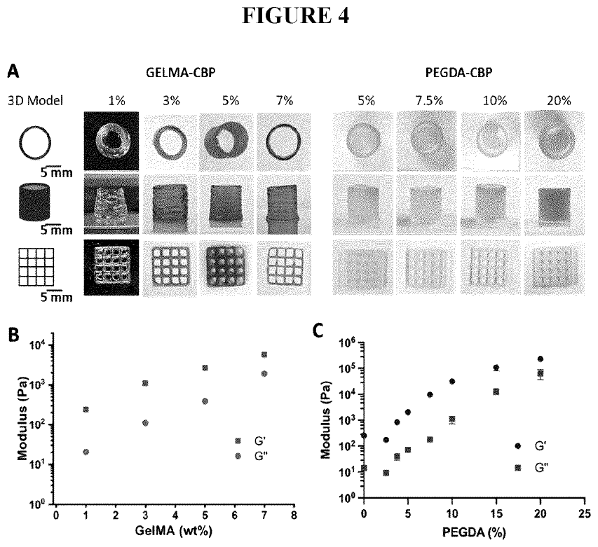

FIG. 4A shows cylinders and mesh-like structures 3D printed using CBP-based inks with different concentrations of PEGDA or GelMA. All the inks presented high printability, and the printed structures supported their own weight, indicating that the PCPAA modifiers are compatible with different concentrations of (bio)polymers. It was observed that PEGDA and GelMA concentrations have a strong influence on the mechanical properties of the crosslinked materials (FIG. 3B-C), where higher concentrations produced higher storage and loss modulus (G′ and G″) for both materials. These results can be explained because a higher PEGDA or GelMA concentration will lead to a higher crosslinking density and the material will be mechanically stronger and more robust.

### Example 4—Extrusion 3D Printing of PCPAA-(Bio)Polymers Prepared with Different Crosslinking Mechanisms

To demonstrate the compatibility of the developed methodology with (bio)polymers presenting different crosslinking mechanisms, formulations with CBP (0.1-1.5%) and PEGDA, XG, or AGA (these polymers were introduced at concentrations in the range of 0.5-25%) were produced. The XG-PCPAA inks were crosslinked using an Fe(III) solution after printing (ionic crosslinking), while AGA-PCPAA inks were crosslinked by decreasing the temperature during the printing process (thermal crosslinking). PEGDA-PCPAA inks were crosslinked by photopolymerization activated via UV or visible light and including LAP in the formulation as a photoinitiator (photo crosslinking). All the formulations showed high printability, providing cylinders and mesh-like structures that retained the designed shape under their own weight. The printability was higher for PEGDA-PCPAA, followed by AGA-PCPAA and XG-PCPAA biomaterial inks. Therefore, the present disclosure shows that using PCPAA for 3D printing is compatible with different crosslinking mechanisms.

Specifically, PEGDA-CBP (10% PEGDA, 0.75% CBP, 0.4% LAP), XG-CBP (1.7% XG, 0.5% CBP), and AGA-CBP (0.7% AGA, 0.5% CBP) were formulated according to the procedure described in Example 1. GelMA-CBP (7% GelMA, 0.5% CBP, 0.5% LAP) was formulated according to the procedure described in Example 2. The inks were 3D printed according to the procedure described in Example 1. The GelMA ink was printed at 37° C.

The XG-CBP ink was crosslinked through the addition of Fe(III) solution after the printing (ionic crosslinking) (FIG. 1B). In the case of the AGA-CBP ink, the constructs were crosslinked by decreasing the temperature during the printing process (thermal crosslinking) (FIG. 1B). PEGDA-CBP and GelMA-CBP inks were crosslinked using visible (405 nm) light and LAP as the photoinitiator (photo crosslinking) (FIG. 4A). Independently of the crosslinking mechanism, the constructs showed good printability and shape fidelity (FIG. 1B, 4A).

### Example 5-Extrusion 3D Printing with Modified and Unmodified Biopolymers that are Non-Extrudable or Exhibit Low Printability

PCPAA can be used to print synthetic polymers such as PEGDA. However, both modified and unmodified biopolymers are of interest for a broad range of bioprinting applications because they can form scaffolds that have improved biocompatibility and maintain/promote cellular metabolism. The present disclosure relates the use of PCPAA for extrusion 3D printing with high printability of non-extrudable or low printability modified and unmodified biopolymers, like XG, AGA, GelMA, whey protein methacrylate (WPMA), or silk fibroin methacrylate (SilkMA). Inclusion of CBP (0.1-1.5%) allowed to obtain printable biomaterial inks with biopolymers in a concentration range of 0.5-30%. All the inks were extrusion 3D printed with good printability and produced structures with excellent fidelity to the designed shapes. The cylinder constructs were able to support their own weight in all cases.

Specifically, XG-CBP (1.7% XG, 0.5% CBP) and AGA-CBP (0.7% AGA, 0.5% CBP) were formulated according to the procedure described in Example 1. GelMA-CBP (5% GelMA, 0.5% CBP, 0.5% LAP) was formulated according to the procedure described in Example 2. SilkMA ink was obtained by mixing the proper volume of 15% SilkMA stock solution with the appropriate amount of 1.5% CBP and 4% LAP stock solutions to obtain a final formulation with 5% SilkMA, 0.5% CBP, and 0.1% LAP. WPMA ink was obtained by mixing the proper volume of 15% WPMA stock solution with the appropriate amount of 1.5% CBP and 4% LAP stock solutions to obtain a final formulation with 7% WPMA, 0.5% CBP, and 0.5% LAP. The inks were 3D printed according to the procedure described in Example 1. The GelMA ink was printed at 37° C.

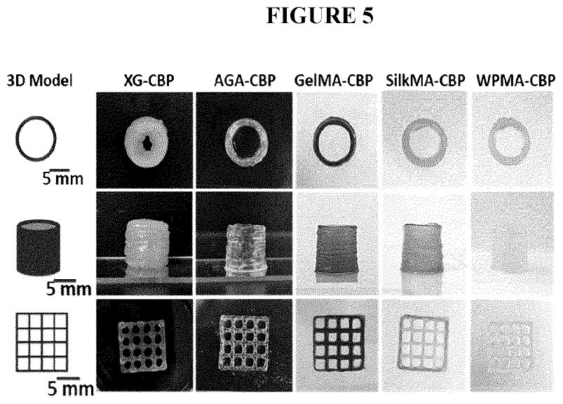

FIG. 5 presents images of the 3D printed constructs obtained using two unmodified biopolymers (XG and AGA) and three modified biopolymers (GelMA, SilkMA, and WPMA). The use of biopolymers for 3D bioprinting applications is of interest due to their biocompatibility and the potential presence in some cases of adhesion points in the crosslinked structures that promote cell adhesion and proliferation. In all cases, the inks showed good printability and shape fidelity, demonstrating that the developed ink platform technology is suitable for the 3D printing biopolymers that are not printable on their own. The introduction of CBP as rheology modifier guarantees that the biopolymer ink formulations have the required rheological properties to extrude properly.

### Example 6-Extrusion 3D Printing with PCPAA-(Bio)Polymers Supplemented with Additives

The present technology can be applied to formulate biomaterial inks with a variety of additives that can contribute to tune and customize the physical, mechanical and biochemical properties of the resulting printed structures according to the intended application. Biomaterial inks containing CBP (0.1-1.5% by wt), PEGDA and additives (e.g., fluorescent microparticles (FM), human lung extracellular matrix (ECM), and cellulose nanocrystals (CNC), LAP, sucrose, PBS, and food dyes) were prepared to showcase the feasibility of including different types of additives within the formulations. The biomaterial inks that incorporated these additives showed good printability and produced structures with excellent fidelity to the designed shape. Overall, such experiments confirm that the methodology to formulate biomaterial inks described herein is compatible with a wide variety of additives.

Specifically, three PEGDA-CBP (7.5% PEGDA, 0.75% CBP, 0.4% LAP) inks, each supplemented with one additive, were formulated according to the procedure described in Example 1. The concentrations of the additives in the final formulations were: human lung tissue-derived ECM (0.4% by wt), cellulose nanocrystals (CNCs at 0.3 and 1%), and fluorescent microparticles (FM at 0.04% by wt). Three GelMA-CBP (3% GelMA, 0.5% CBP, 0.5% LAP) inks, each supplemented with one additive, were formulated according to the procedure described in Example 2. The concentrations of the additives in the final formulations were: CNC (0.1% by wt), FM (0.04% by wt) and collagen (0.1% by wt). The inks were 3D printed according to the procedure described in Example 1. The GelMA ink was printed at 37° C.

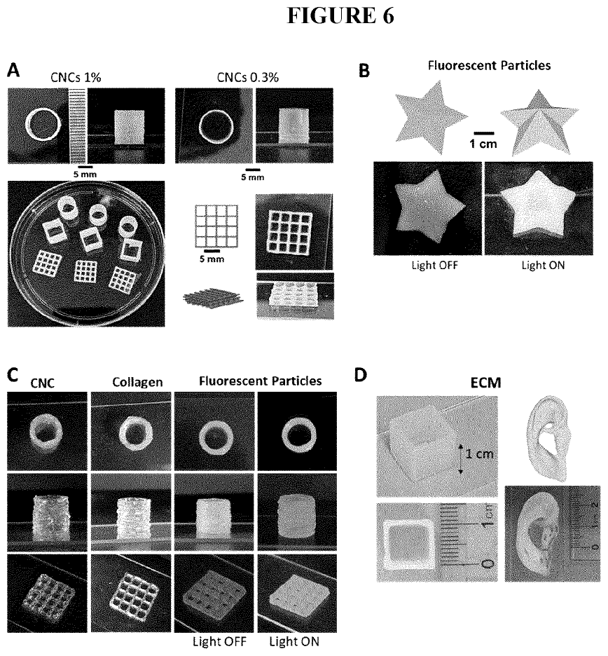

In addition to the physical and mechanical properties of the printed structures, it is also important to have control of the biochemical profile and the composition of the inks, which can be achieved by incorporating additives. The platform ink formulation technology described herein is compatible with the use of a wide variety of additives. For example, FIG. 6 depicts 3D printed constructs with PEGDA or GelMA as crosslinking (bio)polymer and ECM, CNC, collagen, or FM as additives. All the inks presented high printability independently of the additive employed. The 3D printing of cylinder, mesh-like, or even more complex constructs, such as a star or an anatomical-sized ear, demonstrated the robustness of the developed technology when additives from different sources are added to the formulation.

### Example 7—Extrusion 3D Printing of Complex Constructs Made from PCPAA-(Bio)Polymers that are Non-Extrudable or Present Low Printability on their Own

The present disclosure also relates the use of PCPAA to 3D print with high printability complex constructs from non-extrudable or low printability (bio)polymers. Formulations containing CBP (0.1-1.5%) and PEGDA, AGA, or GelMA ((bio)polymer concentration range 0.5-30%) were used to 3D print complex and large designs. PEGDA-PCPAA enabled the printing of a hollow ghost, showing that free-standing structures with overhanging features, which are challenging to print with extrusion 3D bioprinting technology, can be printed. AGA-PCPAA and GelMA-PCPAA were used, respectively, to print an ear and a nose of anatomical dimensions showing high accuracy and fidelity to the designed models.

Specifically, PEGDA-CBP (10% PEGDA, 0.75% CBP, 0.4% LAP) and AGA-CBP (0.7% AGA, 0.5% CBP) inks were formulated according to the procedure described in Example 1. The GelMA-CBP (5% GelMA, 0.5% CBP, 0.5% LAP) ink was formulated according to the procedure described in Example 2. The inks were 3D printed on complex constructs according to the procedure described in Example 1. The GelMA ink was printed at 37° C.

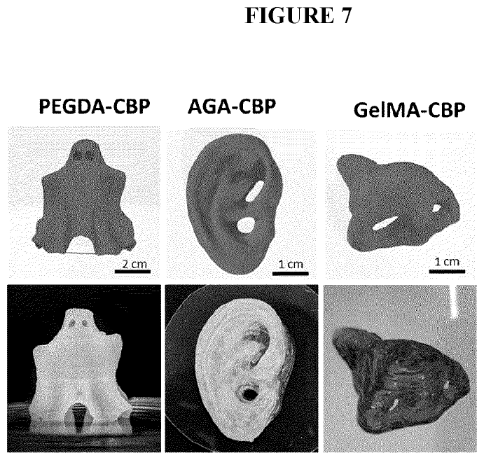

All the tested inks showed good printability and shape fidelity when printing large complex designs (FIG. 7). PEGDA-CBP and AGA-CBP inks enabled the printing of a hollow ghost and an ear of anatomical dimensions, respectively, while a nose was printed using the GelMA-CBP ink. The selected models were challenging for extrusion printing due to the presence of complicated angles, overhanging features, and extended size. In all cases, good fidelity was achieved when compared to the original models. The structures were robust and retained their shape. Additionally, the ghost structure demonstrated that free-standing structures with overhanging features can be printed using CBP as a rheology modifier for (bio)polymers that are non-extrudable or present low printability on their own.

### Example 8—Cell Culture on Top and within Constructs Made from Inks with and without Supplemental Additives

Formulations containing CBP (0.1-1.5%) and PEGDA with or without additives (sucrose, CNC, collagen) or GelMA were employed to culture different cell lines on top and within the constructs. The crosslinkable polymer concentrations used were between 0.5 and 30%. Epithelial cells were seeded on top of PEGDA-PCPAA crosslinked constructs with and without additives and presented excellent viability. Additionally, fibroblasts were cultured within crosslinked PEGDA-PCPAA constructs with and without additives and exhibited excellent viability.

Specifically, human bronchial epithelial cells (cell line HBEC-6KT) were cultured on top of PEGDA-CBP constructs with and without supplemented additives.

PEGDA-CBP (7.5% PEGDA, 0.75% CBP, 0.4% LAP) inks were formulated according to the procedure described in Example 1. PEGDA-CBP-Sucrose (0.2 M) and PEGDA-CBP-Collagen (0.05%) inks were formulated according to the procedure described in Example 6.

HBEC cells were cultured in keratinocyte-SFM medium supplemented with epidermal growth factor (EGF, 0.8 ng/mL), bovine pituitary extract (BPE, 50 μg/mL) and antibiotic-antimycotic (100 U/mL penicillin, 100 μg/mL streptomycin). Puck-like constructs with 10 mm diameter and 1.5 mm height were prepared using PEGDA-CBP, PEGDA-CBP-Sucrose, or PEGDA-CBP-Collagen inks. After crosslinking, the pucks were sterilized with 365 nm UV radiation for 1 h and stored in cell culture media for 48 h in an incubator at 37° C. and 5% CO2 before seeding HBEC-6KT cells on top at a density of 1.5×10⁵ cells per puck. Viability was assessed using a live/dead assay fluorescence staining protocol. All microscopy images were taken using an EVOS M7000 microscope using cubes for excitation/emission of GFP, DAPI, and Texas Red fluorophores. ImageJ was used to generate composite microscopy images by combining the fluorescence channels, and to process the data to calculate cell viability.

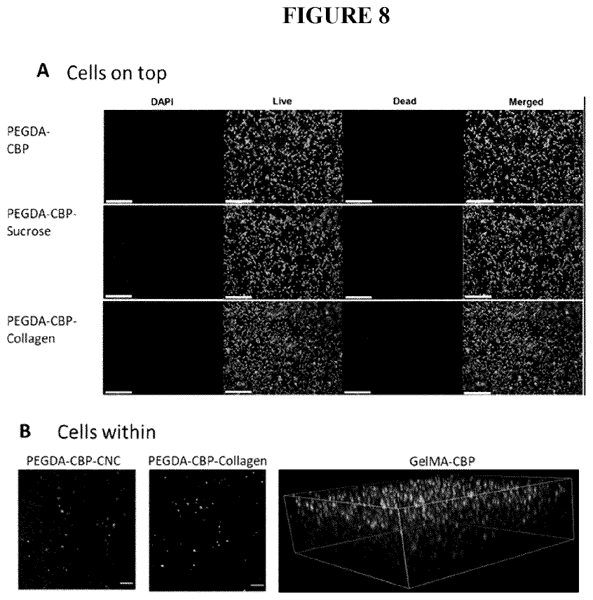

The results showed that the cells on top of the crosslinked materials presented excellent viability (>80% live cells as evaluated by comparison of the panels labeled as “live” and “dead”, and the DAPI channel stains the nucleus of all cells (both live and dead) allowing identification of all cells present in the image for formulations with and without supplemented additives (FIG. 8A). The introduction of additives is relevant for the 3D bioprinting field considering that some materials do not have the desired biochemical cues to modulate cell metabolism. For example, PEGDA does not present adhesion points for the cells and is known to be an antifouling material that is cell-repellent, which can be improved by incorporating additives, such as collagen.

Fibroblast-laden crosslinked constructs (1×10⁶ cells/mL) made of GelMA-PCPAA bioinks were cultured for multiple weeks, showing excellent cell viability (>90%). Overall, the biomaterial ink platform herein described is compatible with the inclusion of a variety of cell lines, with or without the presence of additives that provide important biochemical clues for the normal development of the cells, and with the continuous culture of the included cells for periods of up to several weeks.

Specifically, PEGDA-CBP-CNC (0.3%) and PEGDA-CBP-Collagen (0.05%) inks were formulated according to the procedure described in Example 6 under sterile conditions. Cells were added to the formulation at a density of 5×10⁶ cells/mL while keeping the final volume and the concentration of the rest of the components in the bioink constant. GelMA-CBP (5% GelMA, 0.25% CBP, 0.5% LAP) was formulated according to the procedure described in Example 2 under sterile conditions. Cells were added to the formulation at a density of 1×10⁶ cells/mL while keeping the final volume and the concentration of the rest of the components in the bioink constant.

3T3 fibroblast-laden structures were cultured in DMEM culture media supplemented with 10% fetal bovine serum (FBS) and antibiotic-antimycotic (100 U/mL penicillin, 100 μg/mL streptomycin). Puck-like constructs with 10 mm diameter and 1.5 mm height were prepared by crosslinking the inks with 405 nm visible light. Fluorescence images were acquired using confocal microscopy. Before imaging, samples were labelled with live/dead assay reagents, incubated for 30 min and then washed with phenol-free DMEM supplemented with antibiotic-antimycotic solution. ImageJ was used to generate composite microscopy images by combining the fluorescence channels, and to process the data to calculate cell viability.

The results showed that the cells within the crosslinked materials presented excellent viability (FIG. 8B). In addition, it was possible to culture cells inside the constructs using additives in the formulations, which is, as noted before, of relevance for the 3D bioprinting field.

### Example 9—Extrusion 3D Bioprinting of Bioink Formulations Containing 3T3 Fibroblasts

Formulations containing CBP (0.1%-1.5%), PEGDA or GelMA, and fibroblasts (at 1× 10⁶ or 5×10⁶ cells/mL) were used to 3D print structures with excellent fidelity to the designed models. The (bio)polymer concentrations were between 0.5 and 30%. Cells incorporated into PEGDA-PCPAA or GelMA-PCPAA bioinks were bioprinted into mesh-like constructs to demonstrate the technology's suitability to extrude bioinks containing cells and maintaining their viability. The cells presented excellent viability up to several weeks after the printing process for both PEGDA and GelMA-based inks, demonstrating that the technology herein described is compatible with the 3D bioprinting of cells.

Specifically, PEGDA-CBP (7.5% PEGDA, 0.75% CBP, 0.4% LAP) inks were formulated according to the procedure described in Example 1 under sterile conditions. GelMA-CBP (5% GelMA, 0.4% CBP, 0.5% LAP) inks were formulated according to the procedure described in Example 2 under sterile conditions. The inks were 3D printed according to the procedure described in Example 1. The GelMA ink was printed at 37° C. The 3T3 fibroblasts were cultured according to the procedure described in Example 8.

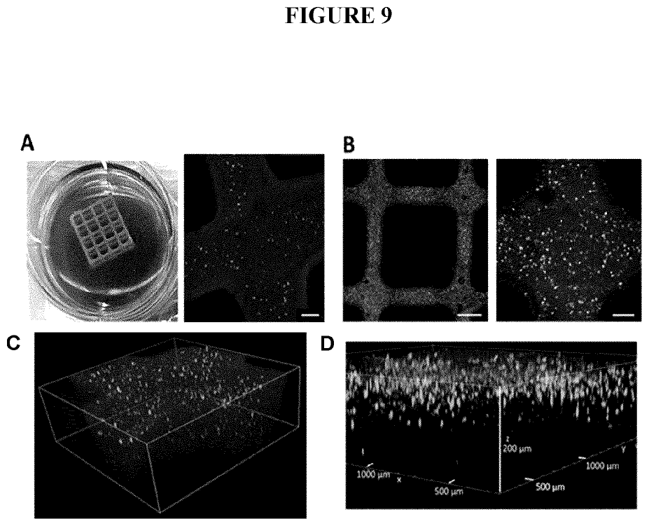

The gel-like properties of the inks were sufficient to maintain the cells encapsulated and well dispersed within the bioink material without settling over time, an advantage that allows execution of long printing workflows (>1 hr) without settling of the cells. The 3D bioprinted structures retained integrity after more than 14 days of culture for both bioinks, with high cell viability (FIG. 9A,B). It was observed that the cells were well dispersed in the three-dimensional structure (FIG. 9C,D), and viable cells could be visualized up to a depth of 300 μm for PEGDA-CBP constructs and almost 1 mm for GelMA-CBP constructs. Overall, the methodology developed herein demonstrated excellent compatibility with 3D bioprinting and excellent cell viability.

### Example 10—Comparison of PCPAA Polymers

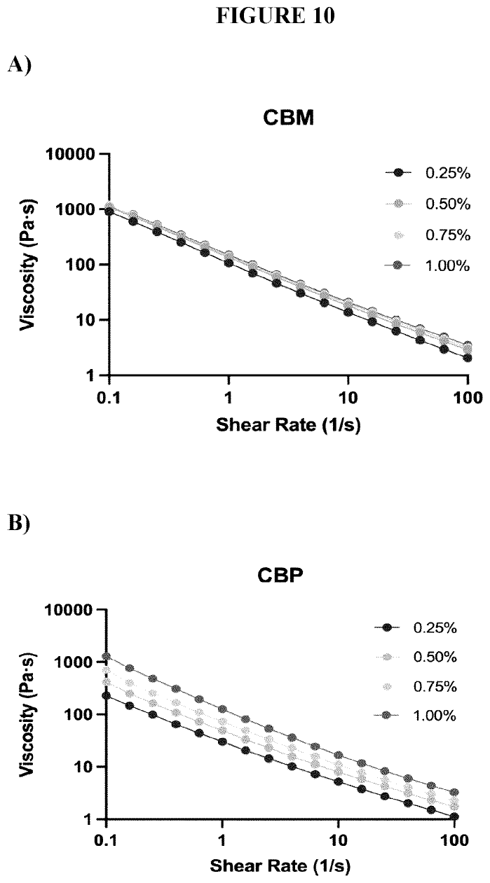

The viscosity of Carbomer 940 (CBM) (homopolymer) and Carbopol EDT 2020 NF®, (CBP) (interpolymer) was compared when subjected to shear stress at different rates. CBM shows near-constant viscosity of 1000 Pa·s at a shear rate of 0.1 s-1 (see FIG. 10A). In contrast, CBP exhibits controlled tuneable viscosity as a function of concentration in the 100-1000 Pa's range (see FIG. 10B). CBP presents rheological behaviour that allows tuning the viscosity of the bioinks containing crosslinkable polymers and additives as a function of the concentration of the rheological modifier. Tuning the viscosity to low values while having shear thinning and thixotropic behaviour is important when including cells within the bioink formulations to support cell viability.

### REFERENCES

[1] A. Schwab, R. Levato, M. D′Este, S. Piluso, D. Eglin, J. Malda, Chem Rev 2020, 120, 11028.
[2] N. Paxton, W. Smolan, T. Bock, F. Melchels, J. Groll, T. Jungst, Biofabrication 2017, 9, 044107.
[3] R. Chang, J. Nam, W. Sun, Tissue Eng Part A 2008, 14, 41.
[4] J. Adhikari, A. Roy, A. Das, M. Ghosh, S. Thomas, A. Sinha, J. Kim, P. Saha, Macromol Biosci 2021, 21, 2000179.
[5] A. G. Tabriz, M. A. Hermida, N. R. Leslie, W. Shu, Biofabrication 2015, 7, 045012.
[6] M. T. Poldervaart, B. Goversen, M. de Ruijter, A. Abbadessa, F. P. W. Melchels, F. C. Öner, W. J. A. Dhert, T. Vermonden, J. Alblas, PLOS One 2017, 12, DOI 10.1371/journal.pone.0177628.
[7] L. E. Bertassoni, J. C. Cardoso, V. Manoharan, A. L. Cristino, N. S. Bhise, W. A. Araujo, P. Zorlutuna, N. E. Vrana, A. M. Ghaemmaghami, M. R. Dokmeci, A. Khademhosseini, Biofabrication 2014, 6, 024105.
[8] S. P. Tarassoli, Z. M. Jessop, T. Jovic, K. Hawkins, I. S. Whitaker, Front Bioeng Biotechnol 2021, 9, 616753.
[9] E. M. Sánchez, J. C. Gómez-Blanco, E. López Nieto, J. G. Casado, A. Macías-García, M. A. Díaz Díez, J. P. Carrasco-Amador, D. Torrejón Martín, F. M. Sánchez-Margallo, J. B. Pagador, Front Bioeng Biotechnol 2020, 8, 776.
D. Wu, Y. Yu, J. Tan, L. Huang, B. Luo, L. Lu, C. Zhou, Mater Des 2018, 160, 486.
Y. J. Shin, R. T. Shafranek, J. H. Tsui, J. Walcott, A. Nelson, D. H. Kim, Acta Biomater 2021, 119, 75.
M. Barreiro Carpio, M. Dabaghi, J. Ungureanu, M. R. Kolb, J. A. Hirota, J. M. Moran-Mirabal, Front Bioeng Biotechnol 2021, 9, 773511.
L. Ning, R. Mehta, C. Cao, A. Theus, M. Tomov, N. Zhu, E. R. Weeks, H. Bauser-Heaton, V. Serpooshan, ACS Appl Mater Interfaces 2020, 12, 44563.
J. Zhao, M. Hussain, M. Wang, Z. Li, N. He, Addit Manuf 2020, 32, 101097.
Y. Jin, A. Compaan, T. Bhattacharjee, Y. Huang, Biofabrication 2016, 8, 025016.
T. Bhattacharjee, S. M. Zehnder, K. G. Rowe, S. Jain, R. M. Nixon, W. G. Sawyer, T. E. Angelini, Sci Adv 2015, 1, e1500655.
V. Ozbolat, M. Dey, B. Ayan, I. T. Ozbolat, Biofabrication 2019, 11, 034101.
D. Baruffaldi, C. F. Pirri, F. Frascella, Bioprinting 2021, 22, e00135.
E. Weber, M. Moyers-González, T. I. Burghelea, J Nonnewton Fluid Mech 2012, 183-184, 14.
P. R. Varges, C. M. Costa, B. S. Fonseca, M. F. Naccache, P. R. de Souza Mendes, Fluids 2019, Vol. 4, Page 3 2019, 4, 3.
Z. Jaworski, T. Spychaj, A. Story, G. Story, Reviews in Chemical Engineering 2022, 38, 881.
M. Babi, R. Riesco, L. Boyer, A. Fatona, A. Accardo, L. Malaquin, J. Moran-Mirabal, ACS Appl Bio Mater 2021, 4, 8443.

1. A bioink composition comprising a rheology modifier, a crosslinking polymer, and, optionally, one or more additives, wherein the bioink composition exhibits printability of at least about 0.4.

2. The bioink composition of claim 1, wherein the rheology modifier comprises one or more partially crosslinked polyacrylic acids.

3. The bioink composition of claim 1 or 2, wherein the rheology modifier is tuneable such that its viscosity decreases with decreasing concentrations of rheology modifier.

4. The bioink composition of any one of claims 1-3, wherein the crosslinking polymer comprises one or more synthetic polymers.

5. The bioink composition of any one of claims 1-3, wherein the crosslinking polymer comprises one or more modified or unmodified biopolymers.

6. The bioink composition of any one of claims 1-4, wherein the synthetic crosslinking polymer is selected from poly(ethylene glycol) diacrylate (PEGDA), poly(olygoethyleneglycol methacrylate) (POEGMA), poly(N-isopropylacrylamide (PNIPAm), polyacrylic acid (PAA), polyacrylamides (PAAm), poly(vinyl alcohol) (PVA), poly(ethylene glycol) (PEG), poly(ethylene oxide) (PEO), poly(2-hydroxyethyl methacrylate) (PHEMA), silicone and mixtures thereof.

7. The bioink composition of any one of claim 1-3 or 5, wherein the unmodified crosslinking biopolymer is selected from proteins, polysaccharides, polynucleotides and mixtures thereof.

8. The bioink composition of claim 7, wherein the unmodified crosslinking biopolymer is selected from collagen (Col), gelatin (Gel), hyaluronan (HA), silk fibroin (SF), whey protein (WP), glycogen (Gly), xanthan gum (XG), agarose (AGA), cell-derived biopolymers, actin, tubulin, tissue-derived extracellular matrix (ECM) proteins or mixtures thereof.

9. The bioink composition of any one of claim 1-3 or 5, wherein the modified biopolymer is selected from acrylated proteins, acrylated polysaccharides and mixtures thereof.

10. The bioink composition of claim 9, wherein the modified biopolymer is collagen methacrylate (ColMA), gelatin methacrylate (GelMA), hyaluronan methacrylate (HAMA), silk fibroin methacrylate (SilkMA), whey protein methacrylate (WPMA), glycogen methacrylate (GlyMA), agarose methacrylate (AGAMA), carboxymethyl cellulose (CMC), carboxymethyl cellulose methacrylate (CMCMA) or a mixture thereof.

11. The bioink composition of any one of claims 1-10, wherein the one or more additives comprise biomolecules; nanoparticles; fibrillar materials; carbon nanotubes;
nanowires; small molecules; dies; mammalian, bacterial, or fungal cells; phages; viral particles; non-ionic molecules to support cell survival; ionic compounds or a combination thereof.

12. The bioink composition of any one of claims 1-11, comprising about 0.1 to about 10% by weight of the rheology modifier and comprising about 0.1-50% by wt of the crosslinking polymer.

13. The bioink composition of claim 12, comprising less than 0.5% by weight of the rheology modifier and a synthetic crosslinking polymer.

14. The bioink composition of any one of claims 1-13, wherein the bioink:
i) is thixotropic;
ii) exhibits shear thinning behaviour such that viscosity decreases at least 100-fold on application of shear; and/or
iii) has a tuneable viscosity in the range of about 1-5000 Pa·s under a shear rate of less than 1 Hz at a pH of 6-8.

15. The bioink composition of any one of claims 1-14, which forms continuous filaments layer-by-layer when extruded from a bioprinter.

16. The bioink composition of claim 1, wherein the rheology modifier and/or the crosslinking polymer are non-extrudable.

17. The bioink composition of claim 1, wherein crosslinking of the crosslinking polymer is activated by application of heat, by chemical crosslinking, by exposure to metal ions or exposure to UV or visible light.

18. The bioink composition of claim 1, wherein the rheology modifier is a partially crosslinked polyacrylic acid interpolymer.

19. Use of a bioink composition according to any one of claims 1-18 for extrusion bioprinting.

20. Use as in claim 19, wherein the extrusion bioprinting is 3D extrusion bioprinting.

21. A structure prepared by extrusion bioprinting with a bioink as defined in any one of claims 1-18, comprising one or more different cell types within the interior of the structure, on the exterior of the structure or a combination thereof.

22. A structure prepared by extrusion bioprinting comprising two or more different bioinks as defined in any one of claims 1-18.

23. A structure prepared by extrusion bioprinting with a bioink as defined in any one of claims 1-18, comprising viable cells.

24. A method of preparing a bioink as defined in any one of claims 1-18, comprising the steps of:
i) combining a rheology modifier with a crosslinking polymer under conditions suitable to prevent gelling of the mixture; and
ii) optionally adding one or more additives to the mixture.

25. A method of extrusion bioprinting comprising the steps of:
i) extruding a bioink prepared by the method of claim 24 to a surface;
ii) exposing the bioink to conditions suitable to promote crosslinking of the crosslinking polymer either before step i) or after step i); and
iii) optionally treating the surface with a stimulus to alter a property of the bioink.

26. The method of claim 25, wherein the crosslinking is activated by application of heat, by chemical crosslinking, by exposure to metal ions or exposure to UV or visible light.

27. The method of claim 25, wherein the stimulus is a mechanical, magnetic, ionic, electric, light, temperature, pH, or oxygen stimulus.
